(19) United States
(12) Patent Application Publication
(10) Pub. No.: US 2001/0051147 A1
(43) Pub. Date: Dec. 13, 2001

(54) Methods for immunostimulation using binding agents for the Fc receptor of immunoglobulin A

(76) Inventor: Jan G.J. van de Winkel, Zeist (NL)

Correspondence Address: LAHIVE & COCKFIELD, 28 STATE STREET, BOSTON, MA, 02109, US

(21) Appl. No.: 09/820,099

(22) Filed: Mar. 27, 2001

Related U.S. Application Data
(60) Provisional application No. 60/192,727, filed on Mar. 27, 2000.

(51) Int. Cl. A61K 39/395; A61K 38/19; A61K 38/20

(52) U.S. Cl. 424/85.1; 424/85.2; 424/155.1; 424/178.1; 424/142.1

(57) ABSTRACT

Methods and compositions for eliminating pathogens from the circulatory system of a subject are discosed. These methods rely on the interaction between monomeric (serum) IgA and FcαRI expressed on liver Kupffer cells. The methods employ therapeutic complexes which are made up of a first portion which specifically binds FcαRI expressed on liver Kupffer cells, or which specifically binds monomeric IgA or the Fc region thereof, linked to a second portion which specifically binds the target cell or antigen.

### BACKGROUND OF THE INVENTION

IgA is abundant in the human body (Kerr, M.A. 1990, Biochem. J 271:285-296). A single class of IgA Fc receptor, FcαRI or CD89, which binds to monomeric IgA has been identified and characterized (Albrechtsen, M. et al., 1988 Immunol. 64:201; Monteiro R., et aL, 1990 J Exp. Med., 171:597). FcαRI is constitutively expressed primarily on cytotoxic immune effector cells including monocytes, macrophages, neutrophils, and eosinophils (Morton, H.C., et aL, 1996 Critical Reviews in Immunology 16:423). FcαRI expression on a sub-population of lymphocytes (Morton, H.C., et al., 1996 Critical Reviews in Immunology 16:423), and on glomerular mesangial cells has been reported (Gomez-Guerrero, C., et al., 1996 J. Immunol. 156:4369-4376). Its expression on monocytes and PMN can be enhanced by TNF-α (Gesl, A., et al., 1994 Scad. J Immunol. 39:151-156; Hostoffer, R.W., et al., 1994, The J. Infectious Diseases 170:82-87), IL-1, GM-CSF, LPS or phorbol esters (Shen L., et al, J. Immunol. 152:4080-4086; Schiller, C.A. et al, 1994, Immunology, 81:598-604), whereas IFN-γ and TGF-β1 decrease FcαRI expression (Reterink, T.J.F., et al., 1996, Clin. Exp. Immunol. 103:161-166). The α-chain of human FcαRI is a heavily glycosylated, type one transmembrane molecule belonging to the Ig super-gene family which also includes receptors for IgG and IgE. One gene located on chromosome 19 encodes several alternatively spliced isoforms of the FcαRI alpha chain (55-110 kDa; Morton, H.C., et al., 1996 Critical Reviews in Immunology 16:423). Myelocytic FcαRI has been shown to be associated with the FcR γ-chain which is implicated as playing a role in FcαRI signal transduction (Morton, H.C. et al. 1995, J. Biol. Chem. 270:29781; Pfefferkom, L.C., et al. 1995, J. Immunol., 153:3228-3236, Saito, K. et al., 1995, J. Allergy Clin. Immunol. 96:1152).

IgA receptors Fcα receptors (FcαR or CD89) are also capable of promoting effector cell function. Binding of ligand to FcαR triggers phagocytosis and antibody mediated cell cytotoxicity in leukocytes and FcαR-bearing cell lines. Fcα receptors can also cooperate with receptors for IgG on effector cells in enhancing the phagocytosis of target cells. Monoclonal antibodies of the IgM (Shen, L. et al., 1989 J. Immunol. 143:4117) and IgG (Monteiro, R.C. et al., 1992 J. Immunol, 148:1764) classes have been developed against FcαR.

FcαRI binds both antigen-complexed and monomeric (serum) IgA1 and IgA2 (Mazangera, R.L. et al., 1990 Biochem. J. 272:159-165), consistent with the receptor being saturated in vivo with monomeric IgA in the same manner as FcγR and FcεRI are saturated with IgG and IgE respectively. Cross-linking FcαRI on myeloid effector cells, by polymeric IgA, IgA immune complexes, or mAb specific for epitopes within or outside the ligand binding domain, stimulates degranulation, superoxide release, secretion of inflammatory cytokines, endocytosis and phagocytosis (Patty, C., A. Herbelin, A. Lihuen, J.F. Bach, and R.C. Monteiro. 1995 Immunology. 86:1-5; Stewart, W.W., R.L. Maz Yegera, L. Shen, and M.A. Kerr. 1994 J. Leucocyte Biology. 56:481-487; Stewart, W.W., and M.A. Kerr. 1990. Immunology. 71:328-334; Shen, L. 1992. J. Leukocyte Biology. 51:373-378.). These physiological responses triggered via FcαRI can be important in the first line of humoral defense on mucosal surfaces (Morton, H.C., M. van Egmond, and J.G.J. van de Winkel. 1996 Critical Reviews in Immunology. 16:423).

Despite the well recognized role for immunoglobulin A (IgA) in mucosal immunity, the function of its receptor, FcαRI (CD89), is poorly understood. FcαRI's capacity to activate leukocytes seems to conflict with the defined anti-inflammatory activity of secretory IgA. A better understanding of the role of this critical receptor in immunity would be of great benefit in the design of improved immunotherapeutics.

### SUMMARY OF THE INVENTION

The present invention is based on the discovery that monomeric (serum) IgA plays a previously unknown important role in systemic immunity by virtue of its interaction with FcαR expressed on liver Kupffer cells and other FcαR-expressing cells (e.g., neutrophils) present at the interface of the mucosal and systemic immune systems (e.g., the sinusoidal lining of the liver). FcαR expressed on these cells selectively binds and causes elimination (e.g., phagocytosis) of monomeric (serum) IgA-antigen complexes by the cells.

Accordingly, in one embodiment, the invention provides a method for eliminating a target cell or antigen from the circulatory system (i.e., the portal circulation) of a subject by administering to the subject a composition (e.g., a molecular complex) comprising a first portion which specifically binds FcαRI expressed on liver Kupffer cells, or which specifically binds monomeric IgA or the Fc region thereof (which, in turn, binds FcαRI), linked to a second portion which specifically binds the target cell or antigen. In certain embodiments, the first portion of the complex binds a site on the FcαR that is distinct from the binding site for IgA, so that binding of the complex is not blocked by endogenous IgA. The first and second portions of the complex can be linked, e.g., by chemical conjugation or by genetic (recombinant) fusion.

In a particular embodiment of the invention, the first portion of the complex comprises serum (monomeric) IgA or a portion thereof (e.g., the Fc portion). In another embodiment, the first portion of the complex comprises an antibody, or fragment thereof, which specifically binds FcαRI or which specifically binds monomeric IgA or the Fc region thereof. Preferred antibodies include human, humanized and single chain antibodies, including Fab fragments thereof.

In another particular embodiment, the second portion of the complex comprises an antibody, or fragment thereof, which specifically binds to the target cell or antigen (e.g., a bacterium, an allergen, a fungus, or a virus). Alternatively, the second portion can be a ligand, e.g., which binds to a receptor on a target cell. For example, the ligand can be a ligand specific for a tumor cell.

The compositions of the present invention can be used to prevent entry of, or eliminate harmful pathogens (e.g., bacteria, viruses, fungi, tumorous cells etc.) from circulation by targeting these pathogens to FcαR-expressing effector cells at the interface (e.g., barrier) of the mucosal and systemic immune systems. In particular, these pathogens can be targeted to FcαR-expressing Kuppfer cells in the sinusoid of the liver which, when bound by the complexes of the invention, mediate phagocytosis of the pathogens. Moreover, FcαR expression on these cells (and other FcαR-expressing cells) can be upregulated by administering cytokines, such as granulocyte/macrophage colony stimulating factor (GM-CSF), interleukin (IL)-6, IL Iβ, IL 8, and tumor necrosis factor (TNF)-α, to the subject (e.g., by injection), thereby enhancing the ability of the cells to bind and to eliminate pathogen FcαR-targeted complexes of the invention. Other particular FcαR-expressing cells which can be targeted are neutrophils which, like liver cells, also selectively bind and phagocytose monomeric (serum) IgA-antigen complexes, but not dimeric (secretory) IgA complexes.

Accordingly, in another aspect, the invention provides a method for eliminating cancerous liver cells (e.g., treating liver cancer) in a subject by targeting cytotoxic agents to FcαRI expressed on the liver cells. This can be achieved by administering to the subject a complex of the invention comprising a first portion which specifically binds FcαRI expressed on the liver cells (e.g., Kupffer cells), or monomeric IgA which binds FcαRI, and a second portion which comprises a cytotoxic (e.g., chemotherapeutic) agent.

In a further aspect, the invention includes a method for treating or preventing septicemia, characterized, for example, by a defective mucosal barrier and concommitantly produced inflammatory mediators, in a subject by administering to the subject a composition (e.g., a molecular complex) of the invention which targets a bacterium, fungus or virus to FcαRI-expressing liver cells. The complex is made up of a first portion which specifically binds FcαRI, or monomeric IgA which binds FcαRI, linked to a second portion which specifically binds the bacterium, virus or fungus.

Other embodiments of the invention will be apparent from the detailed description below.

### BRIEF DESCRIPTION OF THE DRAWINGS

FIG. 1 shows FcαRI expression on Kupffer cells. (a) Paraffin liver sections of G-CSF treated NTg mice (left panel), and untreated (middle panel), or G-CSF treated (right panel) CD89 Tg mice were stained for expression of human FcαRI. Bar represents 30 μm (pictures taken with objective 40x; inset right panel, objective 100x). Only in G-CSF treated Tg mice cytoplasmic staining for FcαRI was found in stellate-shaped cells, lining the liver sinusoids. This experiment was repeated five times with similar results. (b) A double staining for both FcαRI and a macrophage marker (F4/80) was performed to identify stellate cells as Kupffer cells. F4/80 and FcαRI immunoreactivity are shown in blue and red, respectively, as described in the methods section. Kupffer cells of G-CSF treated Tg mice stain positive for FcαRI (right panel; cells are both blue and red), whereas Kupffer cells in NTg mouse livers are negative for FcαRI (left panel; only blue staining). Bar represents 50 μm (objective 20x). (c) Isolated Kupffer cells of G-CSF treated Tg (red line), and NTg control littermates (black line) were stained with Pe-labeled anti-FcaRI mAb A59 (Monteiro, R.C., et al. J. Immunol. 148, 176-1770 (1992)) and analyzed by flow cytometry, showing positive staining of Tg Kupffer cells. (d) Expression of FcαRI on human Kupffer cells. A liver sample of a patient with active viral Hepatitis type C is shown (cryo section). Sections were stained for both CD68, a human macrophage marker (blue), and FcαRI (red) Left panel: negative control (anti-FcαRI Ab omitted). The right panel shows positive Kupffer cells. Bar represents 30 μm (objective 40x; inset, objective 100x).

FIG. 2 shows that Kupffer cells expressing FcαRI mediate phagocytosis of IgA-coated bacteria FITC labeled. Serum IgA opsonized E. coli bacteria were injected i.v. into G-CSF-treated mice. Mice were sacrificed and liver section taken. (a) Fluorescence of NTg (left panel) and Tg (right panel) liver sections was analyzed with fluorescence microscopy. Bar represents 50 μm (objective 20x; inset right panel objective 40x). (b) FITC fluorescence of Tg liver sections was determined (left panel), before staining with F4/80 mAb (middle panel; red) to identify fluorescent stellate cells as Kupffer cells. A computerized overlay picture of both images (right panel) was produced demonstrating fluorescent IgA-coated bacteria to co-localize with F4/80 positive Kupffer cells. Bar represents 50 μm (c) Confocal microscopy pictures showing two different cell layers confirmed that bacteria are phagocytosed by Tg Kupffer cells. Bar represents 15 μm. (d) Numbers of FITC-fluorescent Kupffer cells of NTg (open bar) and Tg (dotted bar) mice were quantitated by microscopy. Data (mean ±SD) are representative of results obtained in three separate experiments. * p<0.01.

FIG. 3 shows that serum IgA mediates phagocytosis via FcαRI in contrast to secretory IgA (SIgA). (a) Non-reduced SDS-PAGE analysis of IgA preparations; lane 1: serum IgA (ICN,), lane 2: SIgA (ICN,), lane 3: SIgA (Sigma). Molecular weight markers are indicated on the left (m) Proteins were stained with Coomassie Brilliant Blue. (b) E coli bacteria were incubated with PBS or IgA preparations, washed and stained with PE-labeled F(ab′)₂ fragments of goat anti-human IgG (left panel) or IgA (right panel) antiserum. Fluorescence was analyzed by flow cytometry, showing all preparations to certain similar amounts of IgA Ab directed against E. coli. Black line; PBS, red line; serum IgA, blue line; SIgA ICN, yellow line; SIgA Sigma (c) NTg or Tg PMN were incubated with FITC-labeled, IgA-opsonized E. coli. bacteria. FITC fluorescence of PMN, reflecting bacterial uptake, was analyzed by flow cytometry. Black line; NTg+serum IgA, yellow line; NTg+SIgA ICN, pink line; NTg+SIgA Sigma. red line; Tg+serum IgA, brown line; Tg+SIgA ICN, blue line; Tg +SIgA Sigma. In addition, Tg PMN were incubated with CD89-blocking mAb My432²¹ prior to incubation with serum IgA-coated bacteria; green line. A representative experiment out of-four is shown. (d-f) Human PMN were incubated with IgA opsonized E. coli bacteria (see c), and analyzed by flow cytometry (d, open bars; without blocking mAb, dotted bars; with blocking mAb My43). Cytospin preparations were analyzed with light (e) or confocal microscopy (f; PMN incubated with serum IgA-coated bacteria: three cell layers). Bar represents 10 μm. (g) G-CSF treated Tg mice were injected i.v. with FITC-labeled, SIgA-(left panel) or serum IgA-(right panel) opsonized E. coli bacteria. Fluorescence of liver sections was analyzed with fluorescence microscopy. Bar represents 50 μm (objective 20x). (h) Respiratory burst activity of human PMN stimulated with serum IgA (red line), SIgA ICN (green line), SIgA Sigma (blue line) or PBS (brown line). This experiment was repeated three times yielding similar results.

FIG. 4 shows a schematic model for the role of IgA in mucosal immunity. Under physiological conditions (left panel) SIgA, as first line of defense, prevents adherence of bacteria to mucosal surfaces. However, in intestinal disease (right panel) characterized by a damaged epithelial barrier, (bacterial) antigens may invade the underlying intestinal wall. In the portal circulation, these pathogens are exposed to, serum IgA. Under these conditions, inflammatory cytokines induce FcαRI-expression on Kupffer cells, which subsequently filter the portal blood by FcαRI-mediated phagocytosis of serum IgA-coated pathogens before further septicaemia may occur. (yellow: hepatocytes, red; micro-organisms, green: activated FcαRI-expressing Kupffer cells, blue/ black Ab in lumen: SIgA, blue Ab in circulation: serum IgA, P: portal vein, H: hepatic vein).

### DETAILED DESCRIPTION OF THE INVENTION

As part of the present invention, it was discovered that, in a transgenic mouse model, inflammatory mediators induce FcαRI expression on Kupffer cells, causing efficient phagocytosis of serum (monomeric) IgA-coated bacteria in vivo. Secretory (dimeric) IgA does not initiate phagocytosis. Therefore, the present invention showed for the first time that serum IgA-FcαRI interactions on Kupffer cells provide a “second line” of defense in mucosal immunity, by eliminating invasive bacteria entering via the portal circulation and thus preventing disease.

In particular, as described in the Examples below, to investigate the role of FcαRI in vivo, a transgenic (Tg) mouse model was created in which the FcαRI cell distribution pattern on blood cells parallels the human situation. A FcαRI-specific immunohistochemical staining was developed to examine expression of FcαRI in tissues. Mice were furthermore injected with IgA-coated bacteria to determine functionality of the receptor.

The results showed that expression of FcαRI is induced on Kupffer cells of Tg mice upon treatment with inflammatory mediators. Human Kupffer cells were also found to express FcαRI. In addition, in vivo challenge of Tg mice with serum IgA-coated E. coli demonstrated efficient phagocytosis of these bacteria by FcαRI-positive Kupffer cells. Secretory IgA (SIgA) did not initiate phagocytosis. This observation was consistent with SIgA's anti-inflammatory nature. Therefore, the studies described herein show that serum IgA-FcαRI interactions on Kupffer cells provide a second line of defense at the interface of mucosal and systemic immunity, by eliminating invasive bacteria entering via the portal circulation, and thus preventing further septicaemic disease.

Receptors for the Fc part of immunoglobulins (FcR) that are expressed on cells of the immune system can trigger a plethora of effector functions upon ligand engagement. Therapeutic binding agents specific for IgA receptors, which can be used in the compositions of the present invention, are described in U.S pat. No. 6,018,031 and copending U.S. application Ser. No. 08/890,011, the complete contents of which are incorporated herein by reference. Extensive research in the last decade has provided considerable insight into the role that Fc receptors for immunoglobulin (Ig) G and IgE play in physiological and pathological events (Ravetch, J.V. Curr. Opin. Immunol. 9, 121-125 (1997); Van de Winkel, J.G.J. & Hogarth, P.M., eds. The immunoglobulin receptors and their physiological and pathological roles in immunity. Kluwer Academic Publishers, Dordrecht, The Netherlands (1998); Kinet, J. -P. Annu. Rev. Immunol. 17, 931-972 (1999)). Relatively little is known, however, about receptors for IgA. This is surprising if one considers that more IgA is produced daily than all other isotypes combined (66 mg/kg/day).

The majority of IgA is expressed in two distinct forms: secretory IgA (SIgA) is the predominant antibody (Ab) in secretions and exists as a dimeric complex containing a joining J chain and secretory component, whereas serum IgA is predominantly monomeric (Kerr, M.A. Biochem. J. 271, 285-296 (1990); Russell, M.W., et al., Biological activities of IgA. in: Mucosal Immunology, eds. P.L. Ogra et al., 225-240 (Academic Press, San Diego, CA, 1998)).

Although several laboratories have demonstrate that serum IgA's capacity to initiate effector functions in vitro (Kerr, M.A. Biochem. J. 271, 285-296 (1990), its in vivo role remains poorly understood. Secretory IgA, in contrast, is considered the major mediator of specific immunity at mucosal surfaces and, as such, provides a “first” line of defense against pathogenic micro-organisms. At the mucosa, it is crucial to maintain immunological responses against foreign pathogens, while responses against commensal bacterial flora and dietary antigens must be avoided. The classical view of the function of SIgA is therefore a passive one; by adhering to microbes in the intestinal lumen, it prevents attachment and invasion of mucous membranes by micro-organism (Mestecky, J, et al. Clin. Immunol. Immunopathol. 40, 105-114 (1986); Mazanec, M.B., et al. Immunol. Today 40, 430-435 (1993); Lamm, M.E. Annu. Rev Microbiol. 51, 311-340 (1997); Brandtzaeg, P. et al. Immunol Today 20, 141-145 (1999)). An additional role of neutralizing viruses intracellularly was described, as well (Mazanec, M.B., et al. Proc. Natl. Acad. Sci. U.S.A 89, 6901-6905 (1992)). SIgA, however, does not trigger deleterious inflammatory responses under physiological conditions.

Phagocytes were nevertheless found to express a receptor for IgA (FcαRI, CD89), that potently triggers activatory responses like phagocytosis, antibody-dependent cellular cytotoxicity (ADCC), and secretion of inflammatory mediators (Maliszawski, C.R., et al. J. Exp. Med. 172, 1665-1672 (1990); Monteiro, R.C., et al. J Exp. Med 171, 597-613 (1990); Morton, H.C., et al. Crit. Rev. Immunol. 16, 423-440 (1996); Kerr. M.A. & Woof, J.M. Fca receptors, in: Mucosal Immunology, eds. P.L. Ogra et al., 213-224 (Academic Press, San Diego, CA, 1998)), which challenges the paradigm of SIgA as a non- or even anti-inflammatory antibody. The biological significance of this receptor, therefore, remains unclear.

Accordingly, the present invention is based on the discovery that FcαRI-expressing Kupffer cells, which represent a crucial cell population at the interface of mucosal and systemic immunity, are capable of mediating efficient phagocytosis of serum IgA-antigen (e.g., baceria) complexes. Furthermore, although both serum IgA and SIgA (though to a much lesser extent) initiated respiratory burst activity, only serum IgA was able to initiate phagocytosis. This is in agreement with a more passive role of SIgA, but attributes a significant function for serum IgA in immunity. Therefore, whereas SIgA's main function is the prevention of bacterial entrance, FcαRI-serum IgA interactions on Kupffer cells provide a second line of defense in mucosal immunity.

In one in one embodiment, the invention provides a method for eliminating a target cell or antigen from the circulatory system (i.e., the portal circulation) of a subject by administering to the subject a composition (e.g., a molecular complex) comprising a first portion which specifically binds FcαRI expressed on liver Kupffer cells, or which specifically binds monomeric IgA or the Fc region thereof (which, in turn, binds FcαRI), linked to a second portion which specifically binds the target cell or antigen. In certain embodiments, the first portion of the complex binds a site on the FcαR that is distinct from the binding site for IgA, so that binding of the complex is not blocked by endogenous IgA. The first and second portions of the complex can be linked, e.g., by chemical conjugation or by genetic (recombinant) fusion.

In a particular embodiment of the invention, the first portion of the complex comprises serum (monomeric) IgA or a portion thereof (e.g., the Fc portion). In another embodiment, the first portion of the complex comprises an antibody, or fragment thereof, which specifically binds FcαR or which specifically binds monomeric IgA or the Fc region thereof.

Preferred antibodies include human monoclonal, humanized and single chain antibodies, including Fab fragments thereof. The term “monoclonal antibody” or “monoclonal antibody composition” as used herein refers to a preparation of antibody molecules of single molecular composition. A monoclonal antibody (mAb) composition displays a single binding specificity and affinity for a particular epitope. Monoclonal antibodies can be prepared using a technique which provides for the production of antibody molecules by continuous growth of cells in culture. These include but are not limited to the hybridoma technique originally described by Kohler and Milstein (1975, Nature 256:495-497; see also Brown et al. 1981 J. Immunol 127:539-46; Brown et al., 1980, J Biol Chem 255:4980-83; Yeh et aL, 1976, PNAS 76:2927-31; and Yeh et al.., 1982, Int. J. Cancer 29:269-75) and the more recent human B cell hybridoma technique (Kozbor et al., 1983, Immunol Today 4:72), EBV-hybridoma technique (Cole et al.., 1985, Monoclonal Antibodies and Cancer Therapy, Alan R. Liss, Inc., pp. 77-96), and trioma techniques.

Tumor specific mAb of human IgA class are not available. Also, it is likely that serum IgA (up to 4.0mg/ml) may interfere with the activity of IgA mAbs under physiological conditions. Another approach employs bispecific antibody molecules to enable FcαRI-dependent cell-mediated cytotoxicity of tumor targets. Bispecific molecules (BsAb) which simultaneously bind to target cells (tumor cells, pathogens) and a trigger receptor (e.g. CD3, CD2, FcγR) on immune effector cells have been described (Michon, J., et al. 1995, Blood, 86:1124-1130; Bakács, T., et aL 1995, International Immunology, 7,6:947-955). BsAbs can be generated from heterohybridomas, or by chemically or genetically linking F(ab′) fragments of two antibodies with different specificities or a F(ab′) fragment and a ligand (Graziano, R.F., et al. 1995, In Bispecific Antibodies. M.W. Fanger, editor. R.G. Landes Company/Austin, TX; Goldstein, J. et al., 1997 J. Immunol 158:872-879). BsAbs produced using a trigger receptor-specific antibody, that binds outside the natural ligand binding domain of the trigger receptor, can circumvent interference by serum antibodies and recruit immune effector cells in the presence of saturating concentration of the natural ligand (Fanger, M. et al., 1989, Immunol. Today, 10,3:92-99). This strategy has been used to produce Fc γR-specific BsAbs, which mediate antibody-dependent cellular cytotoxicity (ADCC) of tumor cells in the presence of monomeric or aggregated IgG (Michon, J., et al 1995, Blood, 86:1124-1130; Bakács, T.,et a. 1995, International Immunology, 7,6:947-955), and have shown promising results in clinical settings, Deo, Y.M., et al, 1997, Immunol Today, 18:127-135. Four FcαRI-specific mAb, identified as A3, A59, A62 and A77, which bind FcαRI outside the IgA ligand binding domain, have been described (Monteiro, R.C. et al, 1992, J. Immunol. 148:1764).

A monoclonal antibody can be produced by the following steps. In all procedures, an animal is immunized with an antigen such as a protein (or peptide thereof) as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. A suspension of antibody-producing cells is removed from each immunized mammal secreting the desired antibody. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line. Mouse lymphocytes give a high percentage of stable fusions with mouse homologous myelomas, however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner are used with to standard techniques, for example, the P3-NS1/1-Ag4-1, P3-x63-Ag8.653 or Sp2/O-Ag14 myeloma lines, available from the American Type Culture Collection (ATCC), Rockville, Md.

The fusion-product cells, which include the desired hybridomas, are cultured in selective medium such as HAT medium, designed to eliminate unfused parental myeloma or lymphocyte or spleen cells. Hybridoma cells are selected and are grown under limiting dilution conditions to obtain isolated clones. The supernatants of each clonal hybridoma is screened for production of antibody of desired specificity and affinity, e.g., by immunoassay techniques to determine the desired antigen such as that used for immunization. Monoclonal antibody is isolated from cultures of producing cells by conventional methods, such as ammonium sulfate precipitation, ion exchange chromatography, and affinity chromatography (Zola et al., Monoclonal Hybridoma Antibodies: Techniques And Applications, Hurell (ed.), pp. 51-52, CRC Press, 1982). Hybridomas produced according to these methods can be propagated in culture in vitro or in vivo (in ascites fluid) using techniques well known to those with skill in the art.

For therapeutic use of antibodies of non-human origin in humans, the non-human “foreign” epitopes elicit immune response in the patient. If sufficiently developed, a potentially lethal disease known as HAMA (human antibodies against mouse antibody) may result. To eliminate or minimize HAMA, it is desirable to engineer chimeric antibody derivatives, i.e., “humanized” antibody molecules that combine the non-human Fab variable region binding determinants with a human constant region (Fc). Such antibodies are characterized by equivalent antigen specificity and affinity of monoclonal and polyclonal antibodies described above, and are less immunogenic when administered to humans, and therefore more likely to be tolerated by the patient.

Chimeric mouse-human monoclonal antibodies (i.e., chimeric antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. 1988 Science 240:1041-1043); Liu et al. 1987 PNAS 84:3439-3443; Liu et al., 1987, J. Immunol. 139:3521-3526; Sun et al. 1987 PNAS 84:214-218; Nishimura et al.., 1987, Canc. Res. 47:999-1005; Wood et al. (1985) Nature 314:446-449; and Shaw et al, 1988, J. Natt Cancer Inst. 80:1553-1559.)

The chimeric antibody can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIIₐ antibody producing hybridoma. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060).

Human mAb antibodies directed against human proteins can be generated using transgenic mice carrying the complete human immune system rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al 1994 Nature 368:856-859; Green, L.L. et al. 1994 Nature Genet. 7:13-21; Morrison, S.L. et al. 1994 Proc. Natl. Acad Sci. USA 81:6851-6855; Bruggeman et al.. 1993 Year Immunol 7:33-40; Tuaillon et al. 1993 PNAS 90:3720-3724; Bruggeman et al.. 1991 Eur J. Immunol 21:1323-1326).

Monoclonal antibodies can also be generated by other methods well known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al. , 1991, Biotechniques 11:152-156). A similar strategy can also been used to amplify human heavy and light chain variable regions from human antibodies (Larrick et al., 1991, Methods: Companion to Methods in Enzymology 2:106-110).

The term “complement” refers to a set of more than 30 serum proteins that are universally present without prior exposure to a particular antigen (see, Liszewski, M. et al., 1993, Fundamental Immunol., 3rd Ed., W. Paul Ed. Ch. 26 “The Complement System” p. 917). The function of the complement system is modification of the membrane of an infectious agent, and promotion of an inflammatory response through cell action. Complement proteins are converted to active forms by a series of proteolytic cleavages. Production of a reactive C3b protein can occur quickly and efficiently via the “classical” complement pathway, or slowly and inefficiently via the “alternative” pathway. C3 is secreted by monocytes and macrophages; a complex of Factors B and D and properdin cleave C3 to yield the products C3a and C3b. These products promote mast cell degranulation, releasing inflammatory molecules such as histamine, proteases, lysozyme, acid hydrolases, and myeloperoxidase. Opsonization of target cell membranes promotes lysis and phagocytosis.

In another particular embodiment of the invention, the second portion of the complex comprises an antibody, or fragment thereof, which specifically binds to the target cell or antigen (e.g., a bacterium, an allergen, a fungus, or a virus). Alternatively, the second portion can be a ligand, e.g., which binds to a receptor on a target cell. For example, the ligand can be a ligand specific for a tumor cell.

The first and second portions of the complex can be linked, e.g., by chemical conjugation using standard techniques well known in the art. Alternatively, they can be genetically expressed as a single (recombinant) fusion construct, also as is well known in the art. Methods for making such “bispecific” complexes, which bind to both FcαR and a second target epitope, including those which include antibodies and antibody fragments as binding reagents, are described in 6,018,031 and U.S. Pat. No. 5,922,845, the entire contents of which are incorporated by reference herein.

The compositions of the present invention can be used to prevent entry of, or eliminate harmful pathogens (e.g., bacteria, viruses, fungi, tumorous cells etc.) from circulation by targeting these pathogens to FcαR-expressing effector cells at the interface (e.g., barrier) of the mucosal and systemic immune systems. In particular, these pathogens can be targeted to FcαR-expressing Kuppfer cells in the sinusoid of the liver which, when bound by the complexes of the invention, mediate phagocytosis of the pathogens. Moreover, FcαR expression on these cells (and other FcαR-expressing cells) can be upregulated by administering cytokines, such as granulocyte/macrophage colony stimulating factor (GM-CSF), interleukin (IL)-6, IL-1β, IL-8, and tumor necrosis factor (TNF)-α, to the subject (e.g., by injection), thereby enhancing the ability of the cells to bind and to eliminate pathogen FcαR-targeted complexes of the invention. Other particular FcαR-expressing cells which can be targeted are neutrophils which, like liver cells, also selectively bind and phagocytose monomeric (serum) IgA-antigen complexes, but not dimeric (secretory) IgA complexes.

As used herein, the term “cytokine” means a protein hormone that can mediate immune defenses against “foreign” substances or organisms. General properties of cytokines are reviewed, for example, by Abbas, A. et al. Cell and Molecular Immunology, 2nd Ed., 1994, Saunders, Philadelphia. Inflammatory cytokines include tumor necrosis factor (TNF), interleukin 1β (IL-1β), IL-6, and γ-interferon (IFN-γ). Production of cytokines by the host can be stimulated by a microbial product, such as lipopolysaccharide (LPS), or by a foreign antigen.

Cytokines can be produced by cells of the immune system, for example, T cells and basophils, and can act on a nearby other cell (paracrine action), or on the producing cell (autocrine action), or can be released into the circulation to act on a distant cell (endocrine). Categories of function of cytokines include: mediation of natural immunity; regulation of lymphocyte activation, growth, and differentiation; regulation of immune-mediated inflammation; and stimulation of leukocyte growth and differentiation.

Cytokine function is initiated by binding to a specific receptor on a target cell. For example, the 17kD TNF polypeptide which functions as a trimer, is produced by phagocytes and T cells. It binds to a specific TNF-receptor located on, for example, a neutrophil or an endothelial cell to activate the responses of inflammation. One such response in these target cells is production of IL-1β, which in turn provokes production of IL-6. Both TNF and IL-1β act on thymocytes to initiate a signal cascade culminating in increased expression of genes encoding Ig proteins. Similarly, IFN-γ binds to specific cell receptors to stimulate expression of different sequences. These cytokines also bind to receptors on liver cells to activate expression of proteins of the acute phase of immune response.

Other cytokines can be anti-inflammatory in their effects on the immune system, for example, IL-4, IL-10, and IL-13 (Joyce, D. et al. 1994, Eur. J. Immunol. 24: 2699-2705; Zurawski, G., et al. 1994, Immunol. Today 15: 19-26). IL-10 thus reduces the pro-inflammatory effects of TNF by down-regulating surface TNF receptor (TNF-R) expression, increasing production of soluble TNF-R, and inhibiting the release of TNF.

Further, the function of human IL-13 protein, studied by stimulation of monocytes with LPS, inhibits production of IL-1α, IL-1β, IL-6, IL-8, MIP-1α, TNF-α, IL-10, GM-CSF and G-CSF. Further, production of IL-1ra (receptor antagonist), a soluble form of the IL-1 receptor, is enhanced. These anti-inflammatory properties are similar to those of IL-4 and IL-10.

Complexes of the present invention can be used in a number of therapeutic applications. In one embodiment, they are used to eliminate cancerous liver cells (e.g., treating liver cancer) in a subject by targeting cytotoxic agents to FcαRI expressed on the liver cells. This can be achieved by administering to the subject a complex of the invention comprising a first portion which specifically binds FcαRI expressed on the liver cells (e.g., Kupffer cells), or monomeric IgA which binds FcαRI, and a second portion which comprises a cytotoxic (e.g., chemotherapeutic) agent.

In another embodiment, the complexes are used to treat or prevent septicemia characterized, for example, by a defective mucosal barrier and concommitantly produced inflammatory mediators, in a subject by administering to the subject a composition (e.g., a molecular complex) of the invention which targets a bacterium, fungus or virus to FcαRI-expressing liver cells. In this embodiment, the composition includes a first portion which specifically binds FcαRI, or monomeric IgA which binds FcαRI, linked to a second portion which specifically binds the bacterium, virus or fungus.

Other uses will be apparent to those of skill in the art from the examples below, which should not be construed as further limiting. The contents of all references, pending patent applications and published patents, cited throughout this application, are hereby expressly incorporated by reference.

### EXAMPLES

Methods

Transgenic mice. Generation of FcαRI mice was described earlier (Van Egmond, M. et al. Blood 93, 4387-4394 (1999)). A 41 kb cosmid clone carrying the FcαRI gene, served as transgenic construct. Mice were bred and maintained at the Transgenic Mouse Facility of the Central Animal Laboratory, Utrecht University, The Netherlands. All experiments were performed according the institutional and national guidelines.

Immunohistochemistry. After deparaffinization of paraffin embedded liver sections, antigen retrieval was performed by incubation with 0.1% pronase (Boehringer Mannheim, Germany) for 8 min. Endogenous peroxidase (PO) was blocked with 1% H₂O₂ in methanol (30 min), and non-specific binding was blocked by incubation with 10% nonnal mouse serum /10°/o normal goat serum. In human livers, excess endogenous biotin was blocked prior to FcαRI staining (Vector Laboratories, Burlingame, Calif.; Blocking kit). Slides were stained for FcαRI, and a mouse macrophage marker with a polyclonal rabbit anti-FcαRI Ab (Westerhuis, R. et al. J. Am. Soc. Nephrol. 10, 770-778 (1999)), and F4/80 mAb (Serotec, Oxford, UK), respectively. Human (cryo) sections were stained with a human macrophage marker CD68 (Dako, Denmark). The anti-FcαRI Ab was detected with a biotinylated goat anti-rabbit antiserum (Vector), and avidin-biotin complex (Dako). Immunoreactivity was visualized with 3,3-diaminobenzidine tetrahydrochloride (DAB), or 3-amino-9-ethyl-carbazole (AEC) (Sigma, St Louis, Montana) resulting in brown, or red staining, respectively. FITC labeled rat anti-acrophage F4/80 mAb and FITC labeled anti-CD68 were detected with alkaline phosphatase (AP)-conjugated Sheep anti-FITC mAb (Boehringer Mannheim) and immunoreactivity was visualized with APblue substrate (25 mg Fast Blue, 12.5 mg Naphtol AS-MX phosphate in 1 ml DMF, 35 mg levamisole and 100 ml TRIS (pH 8.5)). Alternatively F4/80 immunoreactivity detected with PO-conjugated rabbit anti-rat Ab (Dako) and PO-conjugated swine anti-rabbit Ab (Dako). AEC was used as substrate. Slides were counter stained with Mayer's hematoxylin. All stainings were performed at room temperature.

Isolation of Kupffer cells. Kupffer cells were isolated after collagenase retrograde perfusion of livers and subsequent centrifugal elutriation essentially as described 34. Briefly, a canula was inserted into the inferior vena cava and perfusion at 4 ml/min was started. After cutting the portal vein and ligation of the vena cava inferior caudal to the liver, the perfusion rate was increased to 10 ml/min After 10 min, the collagenase buffer (collagenase type IV, 0.25 mg/ml (Sigma) was perfused through the liver for 10 min. Subsequently, the liver was excised, torn carefully, and resuspended in Hank's buffer (0.2% BSA.). Parenchymal cells were removed by differential centrifugation at 50 g. Kupffer and endothelial cells were separated by centrifugal elutriation using a Beckman J2-21 centrifuge equipped with JE-6B rotor at 3250 rpm, eluting at 25 ml, and 70 ml, respectively. After isolation, liver cell fractions (2×10⁵ cells) were incubated with PE labeled anti-FcaRI mAb A59′ (Mazanec, M.B., et al. Immunol. Today 40, 430-435 (1993)), or an IgG1 isotype control (Pharmingen, San Diego, Calif.), washed and analyzed by flow cytometry (FACScan, Becton Dickinson, San Jose, Calif.).

Phagocytosis assay. E. coli bacteria were cultured overnight at 37° C. in Muller Hinton Broth. Bacteria were labeled by incubation with FITC (Sigma) in 0.1 M NaH₂PO₄/Na₂HPO₄, pH 9.6 for 30 min, and opsonized with human serum or secretory IgA (ICN and Sigma; I mg/ml, 30 min, 37° C.). Levels of opsonization were examined with PE-labeled F(ab′)₂ fragments of goat anti-human IgG or IgA antibodies (Southern Biotechnology, Birmingham, Alabama). Bacteria were incubated with PMN (Effector: target ratio (E: T)-1:100) for 30 min at 4° C. Nonbound bacteria were washed away, and samples were transferred to 37° C. for 20 min. Fluorescence of PMN (due to phagocytosis of FITC-labeled E coli) was analyzed by flow cytometry. In an additional set of experiments 2.5×10⁷ bacteria in 100 μI PBS were injected intravenously in G-CSF treated Tg and NTg mice (subcutaneous injection with murine G-CSF, 1.6 μg/mouse/day, for four days). Mice were sacrificed, 30 min after injection of bacteria and livers were collected. Fluorescence of liver sections was determined with (confocal) fluorescence microscopy. Murine G-CSF was generously provided by Dr. J. Andresen (Amgen, Calif.).

Respiratory burst experiments. Polystyrene tubes were coated with 100 μg/ml human serum IgA (ICN) or SIgA (ICN, or Sigma) for 3 hrs at 37° C. After washing thrice with PBS, tubes were blocked with HEPES complete (20mM Hepes pH 7.4, 132 mM NaCl, 6 mM KCI, 1mM MgSO₄, 1.2 mM NaH₂PO₄, 1 mM CaCI₂, 5.5 mM Glucose, 0.5% BSA, 1.5 mM MgC1₂) for 1 hr at 37° C. The luminol-enhanced chemiluminescence method was used for analysis of real time respiratory burst activity (DeChatelet, L R. et al. J. Immunol 129, 1589-1593 (1982)). Human or mouse PMN (2×10⁵/0.2 ml HEPES) were gently centrifuged (400 rpm, 5 min, 4° C.) and placed in a 953 LB Biolumat (Berthold, Wildbad, Germany). Luminol (150 mM) was injected in all tubes, and light emission was recorded continuously for 30 min at 37° C. Tubes blocked with Hepes complete served as control.

Results

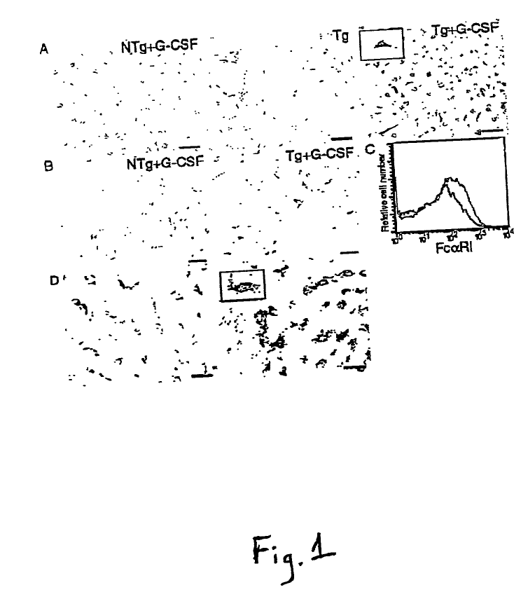

FcαRI expression on Kupffer cells. Because no FcαRI equivalent is presently known in mice, and functional studies are obviously restricted in humans, a FcαRI transgenic (Tg) mouse model was generated to investigate the role of human IgA and its receptor in vivo. FcαRI Tg mice express functional human FcαRI on neutrophils and monocytes, and this model resembles the human situation (Van Egmond, M. et al. Blood 93, 4387-4394 (1999); Van Egmond, M., et al. Immunol. Lett. 68, 83-87 (1999)). Immuohistochemical studies revealed that, except for myeloid cells present in blood and bone marrow, FcαRI was not expressed in Tg tissues. Immunoreactivity of stellate shaped cells in Tg liver samples was observed, however, after treatment of mice with granulocyte colony-stimulating factor (G-CSP) for two days. FcαRI expression on these cells, identified as Kupffer cells, was even more pronounced after four days treatment with G-CSF, whereas Kupffer cells from non-transgenic (NTg) litter mates were negative (Fig. 1a). A double staining for both FcαRI and the mouse macrophage marker F4/80 confirmed the identity of FcαRI-expressing cells as liver macrophages (Kupffer cells). Whereas only F4/80 staining was observed in livers of NTg animals (FIG. 1b, left panel; blue staining), liver samples of Tg mice revealed co-localization of FcαRI and F4/80 immunoreactivity (FIG. 1b, right panel, blue and red staining). Flow cytometric analysis of isolated liver cell fractions confirmed iumunohistochemical data: incubation with PE labeled anti-FcαRI Ab A59 (Monteiro, R.C., et al. J. Immunol 148, 176-1770 (1992)), demonstrated only Kupffer cells of Tg mice to express FcαRI, whereas endothelial cells and hepatocytes were negative (FIG. 1c, and data not shown). Kupffer cells of neither NTg, nor Tg animals did bind an irrelevant IgG isotype control monoclonal Ab (mAb) (data not shown). Importantly, immunohistochemical studies of patient liver biopsies revealed also human Kupffer cells to express FcαRI, supporting this mouse model to be representative of the situation in man (FIG. 1d).

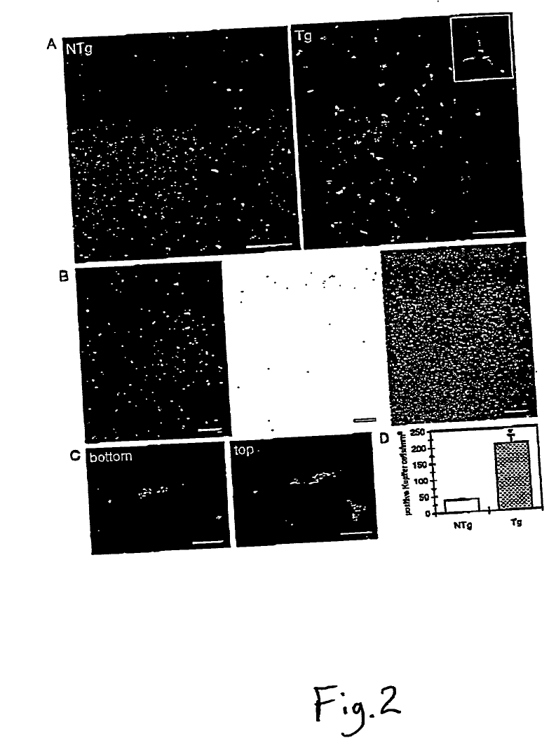

FcαRI-expressing Kupffer cells phagocytose serum IgA-coated bacteria. To assess functionality of Kupffer cell FcαRI, FITC-labeled and serum IgA4; coated E coli bacteria were injected into G-CSF-treated Tg mice and NTg littermates. After 30 minutes, mice were sacrificed and liver samples taken. Fluorescence microscopy showed fluorescent cytoplasm of stellate cells in Tg liver sections, indicating that these cells had ingested bacteria. In comparison, livers of NTg mice showed a five-fold reduction in fluorescence (FIG. 2a and d). To verify identity of these stellate shaped phagocytic cells, fluorescence of slide sections was defined, prior to macrophage staining with F4/80 (FIG. 2b). After staining, immunoreactivity of coordinated sections was examined. Computer overlays demonstrated co-localization of FITC-labeled, serum IgA-coated bacteria with F4/80 positive cells, confirming the sessile macrophage nature of the phagocytic cells and their identify as Kupffer cells. (FIG. 2b. right panel). Furthermore, confocal microscopic analysis of Tg Kupffer cells revealed serum IgA-coated bacteria to be ingested (FIG. 2c), indicating phagocytosis mediated via FcαRI.

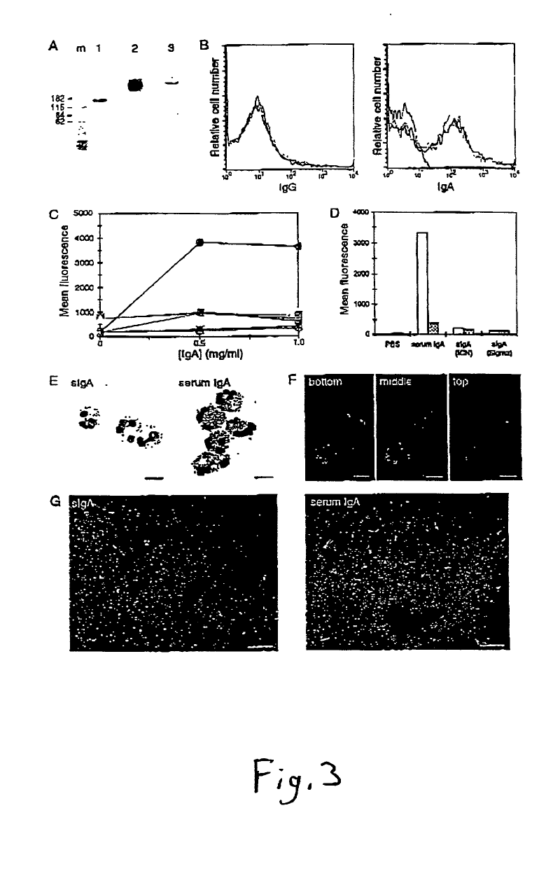

Serum IgA, but not secretory IgA initiates FcαRI- mediated phagocytosis. Despite increasing interest in this area, interactions of secretory versus serum IgA with effector cells remain poorly understood. Several conflicting reports describe either the ability or disability of SIgA to trigger functions like phagocytosis (Kerr, M.A. Biochem. J. 271, 285-296 (1990); Weisbart, R.H, et al. Nature 332, 647-648 (1988); Nikolova, E.B. etal. J Leukoc. Biol. 57, 875-882 (1995); Gorter, A. et al. Immunology 61, 303-309 (1987)). Well-defined and commercially available serum and SIgA preparations showed similar binding ability to E coli bacteria, while no contamination with IgG Ab was detectable (FIG. 3a and b). HPLC analyses demonstrated serum IgA to be mainly monomeric (<5% dimeric IgA, no polymeric IgA), whereas both SIgA preparations consisted of dimeric IgA (no detectable monomeric or polymeric IgA). Incubation of polymorphonuclear cells (PMN), from either Tg mice or humans with serum IgA-opsonized bacteria efficiently initiated phagocytosis, which was blocked by preincubation with mAb, a mAb recognizing the FcαRI IgA binding site (Shen L., et al. J. Immunol. 143, 4117-4112 (1989)). SIgA was unable to initiate phagocytosis (FIG. 3c-f), and PMN of NTg mice did not exhibit phagocytosis of either serum-or SIgA-coated bacteria. The observation that PMN were unable to phagocytose SIgA-coated bacteria was confirmed by experiments with V-gene matched chimeric serum-and SIgA antibodies directed against PorA of group B meningococci. Only serum IgA induced PMN-mediated phagocytosis of bacteria, whereas SIgA was inactive (Vidarsson et al., manuscript submitted).

Tg and NTg PMN had similar capacities to ingest IgG coated E. coli bacteria. Experiments with IgG-coated bacteria, furthermore, documented IgA to be at least as effective as IgG-initiating phagocytosis of E coli (data not shown). Ingestion of unopsonized E. coli was far less efficient (FIG. 3c and FIG. 3d.)

Injection of serum-or SIgA-opsonized bacteria into G-CSF-treated Tg mice confirmed the in vitro data: more effective phagocytosis is observed when serum IgA-coated bacteria were injected compared with injection of SIgA-opsonized E. coli (FIG. 3g).

To investigate whether the inability of SIgA to mediate phagocytosis was attributable to defective interaction with FcαRI, the capacity of serum IgA and SIgA to induce a respiratory burst in PMN serum and SIgA was studied, coated to plastic did both induce oxygen radical production in human PMN, which was inhibited by preincubation of cells with the FcαRI-blocking mAb My43. Although the levels of oxygen radical production were comparable after 30 minutes, kinetics of respiratory burst were different between the IgA types (FIG. 3h). Serum IgA induced a rapid respiratory burst, reaching maximal levels by 5 minutes. SIgA, in contrast, triggered delayed oxygen radical production reaching maximal levels only after 20 minutes. In addition, serumand SIgA triggered a respiratory burst in FcαRI Tg PMN (data not shown).

Discussion

Although it is well recognized from in vitro studies that FcααRI represents a potent trigger molecule for phagocytosis, ADCC, and release of inflammatory mediators (Morton, H.C., et al. Crit. Rev. Immunol. 16, 423-440 (1996); Kerr. M.A. & Woof, J.M. Fcα receptors, in: Mucosal Immunology, eds. P.L. Ogra et al., 213-224 (Academic Press, San Diego, CA, 1998)), its in vivo role is difficult to envisage, since the (secretory) IgA ligand is considered an anti-inflammatory antibody (Mestecky, J, et al. Clin. Immunol. Immunopathol. 40, 105-114 (1986); Mazanec, M.B., et al. Immunol. Today 40, 430-435 (1993); Lamm, M.E. Annu. Rev Microbiol. 51, 311-340 (1997); Brandtzaeg, P. et al. Immunol. Today 20, 141-145 (1999); Russell, M.W., et al. Biochem. Soc. trans. 25, 466-470 (1997)). To resolve this dilemma, we created an FcαRI Tg mouse model to study the role of human IgA and its receptor in vivo. Although FcαRI was not expressed in tissues from Tg mice, treatment with G-CSF induced FcαRI expression on liver Kupffer cells. Previous studies demonstrated FcαRI expression to be under strict regulation by cytokines, indeed. Granulocyte/macrophage colony stimulating factor (GM-CSF). interleukin (IL)-6, IL-1β, IL-8 and tumor necrosis factor (TNF)-α were reported to enhance PMN-or monocytes FcαRI levels (Morton, H.C., et al. Crit. Rev. Immunol. 16, 423-440 (1996); Weisbart, R.H, et al. Nature 332, 647-648 (1988); Nikolova, E.B. et al. J. Leukoc. Biol. 57, 875-882 (1995); Shen, L., Collins, et al. J. Immunol. 152, 4080-4086 (1994)), whereas GM-CSF and TNF-α induced expression on Tg macrophages (Van Egmond, M., et al. Immunol. Lett. 68, 83-87 (1999). Alternatively. injection of G-CSF might result in activation of Kupffer cells (Wisse, E. et al. Toxicol. Pathol. 24, 100-111(1996)). Substances like colony stimulating factor (CSF), macrophage-colony stimulating factor (M-CSF), platelet-activating factor, Zymosan and endotoxin were shown to activate Kupffer cells 24, with subsequent secretion of inflammatory mediators, including interleukins and TNF-α (Declcer, K. Eur. J. Biochem. 192, 245-261 (1990)). These latter cytokines might be responsible for the observed effect on FcαRI expression (Morton, H.C., et al. Crit. Rev. Immunol. 16, 423-440 (1996); Kerr. M.A. & Woof, J.M. Fcα receptors, in: Mucosal Immunology, eds. P.L. Ogra et al., 213-224 (Academic Press, San Diego, CA, 1998); Hostoffer, R.W., et al. J Infect. Dis. 170, 82-87 (1994)). Our observation that injection of TNF-α for two days triggers expression of FcαR1 on Kupffer cells supports an indirect effect of G-CSF (data not shown).

Kupffer cells are located in the sinusoidal lining of the liver and have extensive phagocytic, pinocytic and digestive capacity. They are, therefore, believed to guard the liver sinusoids against potential obstruction by debris, but even more importantly, Kupffer cells filter the portal blood of invasive micro-organisms, and play a crucial role in the prevention of septicaemia (Wisse, E. et al. Toxicol Pathol 24, 100-111 (1996)). Since FcαRI-expressing Kupffer cells were shown capable of efficient phagocytosis of serum IgA-coated bacteria, a role for IgA-FcαRI interactions in this process is implied. Only serum IgA, but not SIgA can initiate phagocytosis, which is supported by earlier data of Nikolova et al. (Nikolova, E.B. et al. J Leukoc. Biol. 57, 875-882 (1995)), Shen et al. (Shen, L. et al. Immunology 68, 491-496 (1989)), and Avery et al. (Avery, V.M. et al. Eur. J Clin. Microbiol Infect. Dis. 10, 1034-1039 (1991)). This is not due to absence of interaction between SIgA and FcαRI, since RMN respiratory burst activity can be induced by both serum IgA and SIgA (FIG. 36). Respiratory bursts of- equal intensity were observed after 30 minutes, which is compatible with earlier reports of Gorter et al. (Gorter, A. et al. Immunology 61, 303-309 (1987)), and Shen et al. (Shen, L. et al. Immunology 68, 491-496 (1989)), indicating that SIgA can interact with FcαRI (Stewart, W.W., et al. Immunology 71, 328-334 (1990)). However, kinetics of burst activity were very different between serum IgA and SIgA, and the latter ligand triggered only delayed superoxide production.

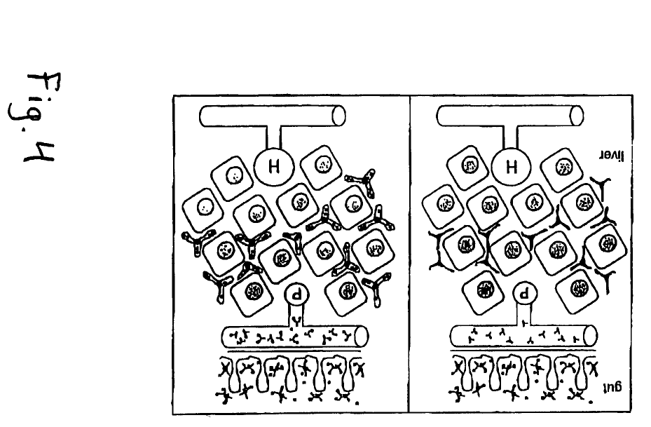

As schematically represented in FIG. 4, although it has been shown that SIgA can trigger respiratory burst activity in vitro (though to a lesser extend than serum IgA), SIgA is unable to mediate phagocytosis, either in vitro or in vivo. Therefore, that the (generally accepted) main function of SIgA is to serve as an “antiseptic coating” of the mucosal wall by preventing adherence and invasion of micro-organisms (FIG. 4, left panel). However, under pathological circumstances in the intestinal tract, characterized by a defective mucosal barrier and concomitantly produced inflammatory mediators, FcαRI expression is induced on Kupffer cells (right panel). Under these conditions Kupffer cells play an important role in maintaining homeostasis by clearance of bacteria from the portal blood, a/o by FcαRI- mediated phagocytosis of serum IgA-coated microorganisms before further septicemia and disease can occur.

In conclusion, there is a dichotomy between the biological roles of serum and secretory IgA. While the main function of SIgA may be to prevent microbiological invasion of the body, serum IgA triggers FcαRI-mediated phagocytosis by blood and liver effector cells. In this way FcαRI-positive Kupffer cells serve as “second line” of defense in mucosal immunity, by eliminating invasive pathogens from the portal and systemic circulation. Importantly, FcαRI was recently identified as potent trigger molecule for antibody-based cancer immunotherapies in vitro (Valerius, T. et al. Blood 90, 4485-4492 (1997); Deo, Y.M., et al. J. Immunol. 160, 1677-1686 (1998)). Even more, treatment of B cell lymphoma-bearing FcαRI Tg mice with bispecific antibody, targeting FcαRI and turmoridiotype, resulted in potent anti-tumor effects in vivo (van Egmond and Glennie, unpublished data). Since Kupffer cells exhibit prominent cytotoxicity against tumor cells, it is possible to mobilize this cytotoxic capacity for immunotherapy of primary and metastatic malignancies in the liver.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for eliminating a target cell or antigen from the circulatory system of a subject comprising, administering to the subject a complex comprising a first portion which specifically binds FcαRI expressed on liver Kupffer cells, or which specifically binds monomeric IgA or the Fc region thereof, linked to a second portion which specifically binds the target cell or antigen.

2. The method of claim 1, wherein the first portion of the complex binds a site on FcαR that is distinct from the binding site for IgA, so that binding of the complex is not blocked by IgA.

3. The method of claim 1, wherein the first portion of the complex comprises monomeric IgA or the Fc region thereof.

4. The method of claim 1, wherein the first portion of the complex comprises an antibody or antibody fragment which specifically binds FcαRI.

5. The method of claim 4, wherein the antibody is a human or humanized antibody.

6. The method of claim 1, wherein the second portion of the complex comprises an antibody or an antibody fragment thereof which specifically binds the target cell or antigen.

7. The method of claim 1, wherein the target cell is a cancer cell.

8. The method of claim 1, wherein the target antigen is selected from the group consisting of a bacteria, a virus, and a fungus.

9. The method of claim 1, further comprising the step of administering to the subject a cytokine which increases expression of FcαRI on Kupffer cells.

10. The method of claim 9, wherein the cytokine is selected from the group consisting of GM-CSF, IL-6, IL-1β, IL-8, and TNF-α.

11. The method of claim 1, wherein the complex is administered by injection.

12. The method of claim 11, wherein the complex is administered intraveneously.

13. A method for treating liver cancer in a subject comprising, administering to the subject a complex comprising a portion which specifically binds FcαRI expressed on liver cells, or which specifically binds monomeric IgA or the Fc region thereof, linked to a cytotoxin.

14. The method of claim 13, wherein the portion which binds FcαRI comprises monomeric IgA or the Fc region thereof.

15. The method of claim 13, wherein the portion that binds FcαRI comprises an antibody or antibody fragment.

16. The method of claim 15, wherein the antibody is a human or humanized antibody.

17. The method of claim 1 1, further comprising administering to the subject a cytokine which increases expression of FcαRI on the liver cells.

18. The method of claim 17, wherein the cytokine is selected from the group consisting of GM-CSF, IL-6, IL-1β, IL-8, and TNF-α.

19. A method for treating or preventing septicemia in a subject comprising administering to the subject a complex comprising a portion which specifically binds FcαRI expressed on liver cells, or which specifically binds monomeric IgA or the Fc region thereof, linked to a cytotoxin.

20. The method of claim 19, wherein the portion which FcαRI comprises monomeric IgA or the Fc region thereof.

21. The method of claim 19, wherein the portion that binds FcαRI comprises an antibody or antibody fragment.

22. The method of claim 21, wherein the antibody is a human or humanized antibody.

23. The method of claim 19, further comprising administering to the subject a cytokine which increases expression of FcαRI on the liver cells.

24. The method of claim 23, wherein the cytokine is selected from the group consisting of GM-CSF, IL-6, IL-1β, IL-8, and TNF-α.
